(19) United States
(12) Patent Application Publication
(10) Pub. No.: US 2018/0079119 A1
(43) Pub. Date: Mar. 22, 2018

(54) Housing and Drug Delivery Device herewith and Method for Producing a Housing

(71) Applicant: Sanofi, Paris (FR)

(72) Inventors: Anthony Paul Morris, Coventry West Midlands (GB); William Marsh, Buckinghamshire (GB); Matthew Jones, Warwick,Warwickshire (GB); Samuel Steel, Warwickshire (GB)

(21) Appl. No.: 15/517,377

(22) PCT Filed: Oct. 9, 2015
(86) PCT No.: PCT/EP2015/073419

§ 371 Date: Apr. 6, 2017

(30) Foreign Application Priority Data
Oct. 9, 2014 (EP) 14306585.2

(51) Int. Cl. B29C 45/16; A61M 5/24; A61M 5/315; A61M 5/20; B29C 45/02

(52) U.S. Cl. CPC B29C 45/16; A61M 5/24; A61M 5/31515; A61M 5/31553; A61M 5/3158; A61M 2005/3126; A61M 5/2033; B29C 45/02; A61M 2207/00; A61M 2205/583; A61M 5/31561

(57) ABSTRACT

The present disclosure is generally directed to a drug delivery device for selecting and dispensing a number of user variable doses of a medicament and to a housing for such a device. The housing comprises a twin-shot injection moulded casing with at least one window and at least one interface for engaging a further component part of the drug delivery device. The casing comprises a first shell injection-moulded in a translucent material and a second shell injection-moulded in an opaque material. Further, the disclosure is directed to a method for producing such a housing.

### CROSS-REFERENCE TO RELATED APPLICATIONS

This application is a U.S. national stage application under 35 USC § 371 of International Application No. PCT/EP2015/073419, filed on Oct. 9, 2015, which claims priority to European Patent Application No. 14306585.2 filed on Oct. 9, 2014, the entire contents of which are incorporated herein by reference.

### TECHNICAL FIELD

The present disclosure is generally directed to a housing for a drug delivery device and to a drug delivery device for selecting and dispensing a number of user variable doses of a medicament comprising such a housing. Further, the disclosure is directed to a method for producing a housing for a drug delivery device.

### BACKGROUND

Pen type drug delivery devices have application where regular injection by persons without formal medical training occurs. This may be increasingly common among patients having diabetes where self-treatment enables such patients to conduct effective management of their disease. In practice, such a drug delivery device allows a user to individually select and dispense a number of user variable doses of a medicament. The present disclosure is not directed to so called fixed dose devices which only allow dispensing of a predefined dose without the possibility to increase or decrease the set dose.

There are basically two types of drug delivery devices: resettable devices (i.e., reusable) and non-resettable (i.e., disposable). For example, disposable pen delivery devices are supplied as self-contained devices. Such self-contained devices do not have removable pre-filled cartridges. Rather, the pre-filled cartridges may not be removed and replaced from these devices without destroying the device itself. Consequently, such disposable devices need not have a resettable dose setting mechanism. The present disclosure is directed to reusable devices which allow resetting of the device and a replacement of a cartridge. Resetting of the device typically involves moving a piston rod or lead screw from an extended (distal) position, i.e. a position after dose dispensing, into a more retracted (proximal) position.

These types of pen delivery devices (so named because they often resemble an enlarged fountain pen) generally comprise three primary elements: a cartridge section that includes a cartridge often contained within a housing or holder; a needle assembly connected to one end of the cartridge section; and a dosing section connected to the other end of the cartridge section. A cartridge (often referred to as an ampoule) typically includes a reservoir that is filled with a medication (e.g., insulin), a movable rubber type bung or stopper located at one end of the cartridge reservoir, and a top having a pierceable rubber seal located at the other, often necked-down, end. A crimped annular metal band is typically used to hold the rubber seal in place. While the cartridge housing may be typically made of plastic, cartridge reservoirs have historically been made of glass.

The needle assembly is typically a replaceable double-ended needle assembly. Before an injection, a replaceable double-ended needle assembly is attached to one end of the cartridge assembly, a dose is set, and then the set dose is administered. Such removable needle assemblies may be threaded onto, or pushed (i.e., snapped) onto the pierceable seal end of the cartridge assembly.

The dosing section or dose setting mechanism is typically the portion of the pen device that is used to set (select) a dose. During an injection, a spindle or piston rod contained within the dose setting mechanism presses against the bung or stopper of the cartridge. This force causes the medication contained within the cartridge to be injected through an attached needle assembly. After an injection, as generally recommended by most drug delivery device and/or needle assembly manufacturers and suppliers, the needle assembly is removed and discarded.

A further differentiation of drug delivery device types refers to the drive mechanism: There are devices which are manually driven, e.g. by a user applying a force to an injection button, devices which are driven by a spring or the like and devices which combine these two concepts, i.e. spring assisted devices which still require a user to exert an injection force. The spring-type devices involve springs which are preloaded and springs which are loaded by the user during dose selecting. Some stored-energy devices use a combination of spring preload and additional energy provided by the user, for example during dose setting.

### SUMMARY

The housing of known drug delivery devices is often a tubular casing which may include a window and/or an interface for engaging further component parts of the device. For example, EP 1 603 611 B1 discloses a housing having a helical rib (thread) for engaging a groove (thread) of a dose dial sleeve. In most devices, the housing is made from one single material. In contrast to that, EP 1 007 115 B1 discloses a housing with a rigid base and a soft-touch layer formed via a two-shot molding or a co-injection molding process. This fabrication technique permits a chemical bonding to occur between the materials of the base and the soft-touch layer. This housing is provided with a window in the form of an aperture in the casing which is covered by a clear plastic cover.

Attachment of a separate clear plastic cover requires additional assembly effort. Further, especially if a clear plastic cover is to be attached from the outside of a housing, it may be difficult to provide a reliable fixture. Certain aspects of the present disclosure provide an improved drug delivery device and a respective housing including a translucent window.

According to the disclosure, a housing for a drug delivery device comprises a twin-shot injection moulded casing with at least one window and at least one interface for engaging a further component part of the drug delivery device, wherein the casing comprises a first shell injection-moulded in a translucent or transparent material and a second shell injection-moulded in an opaque material. Twin-shot injection moulding is a technique where a first portion of a component part, e.g. the first shell, is injection moulded and the resulting part is used in a further injection moulding step for application of at least one further layer, e.g. the second shell, on the first portion of the component part. This includes examples where the first portion is fully or partly covered by the further layer internally and/or externally. In addition, the further layer may fill gaps, apertures or the like in the first portion, e.g. a rib made from the further layer and extending through the first portion. Preferably, the first shell and the second shell are permanently joined or adhered with each other.

In other words, this disclosure relates to the design of a housing for an injector mechanism including window elements for viewing internal parts, in order to give visual feedback to the user. The windows may include a lens to magnify the visual feedback, allowing it to be viewed more easily by visually impaired users.

According to a further aspect of the disclosure, at least one, preferably two, lens is formed by the translucent or transparent material, for example at the distal end of the housing, to indicate dose setting and/or dose dispensing.

A further aspect of the disclosure refers to a housing for a drug delivery device having a distal end and an opposite proximal end with the proximal end face of the housing being provided with axially extending teeth or splines for rotationally constraining a further component part, e.g. a dial grip or a trigger button, to the housing depending on the axial position of the component part relative to the housing. The teeth may be saw teeth with same angles on both faces or with different angles, i.e. steeper faces on one side and shallower faces on the opposite side.

If the second shell at least partly surrounds the first shell, the interior of the housing is hidden from outside by the opaque second shell in such regions of the housing, which are covered by the second shell. On the other hand, openings in the second shell may be used to define a window or lens made by providing the first shell. Thus, this technique is especially useful if more than one single window or lens is to be provided in a housing.

In a preferred embodiment of the disclosure, the first shell comprises the at least one window and the at least one interface for engaging a further component part of the drug delivery device. In addition or as an alternative, the second shell comprises at least one interface for engaging a further component part of the drug delivery device. The at least one interface may have any suitable form for engaging, guiding, holding or constraining a further component part of the drug delivery device. The interface preferably comprises a thread, a rib, a groove, a bead, a tooth, a ramp and/or an arm. Further, the interface may be suitable for permanent interaction with a component part for temporary interaction, for example a clutch which is coupled to and decoupled from the housing.

The first shell may comprise a tubular portion with an axially extending protrusion at the distal end and a series of crown teeth at the opposite, proximal end. Such crown teeth may be used to rotationally lock a component part, e.g. a button, to the housing if the teeth engage with corresponding teeth on this component part. Each tooth may have one angled face and one flat face. As an alternative, both faces may be angled. Preferably, the tubular portion comprises an oblong elevation extending in the axial direction, which forms the at least one window. In an embodiment, the oblong elevation may be framed and/or partly covered by the second shell to define more than one window region. Further, the first shell may comprise an insert, e.g. located at or near the distal end of the tubular portion.

The second shell may comprise ramps, e.g. for attachment of a cap, and at least one guiding rib or groove, e.g. for attachment of a cartridge holder of the device.

A drug delivery device for selecting and dispensing a number of user variable doses of a medicament comprises a housing as mentioned above, a cartridge holder and a cartridge containing a medicament.

Preferably, the housing is coupled via the at least one interface to at least one of the cartridge holder, a piston rod, a drive member, a nut, a dose setting element, a button, a dose setting grip, a drive spring, a gauge element, a clutch and/or a clutch spring. In an exemplary embodiment the piston rod is in threaded engagement with the housing, preferably with the first shell. The drive member may be axially displaceable relative to the housing and may comprise teeth engaging with corresponding teeth of the housing, preferably of the first shell, depending on the relative axial position of the drive member to the housing. Preferably, the dose setting element is clipped to the housing, e.g. to the first shell, to allow relative rotational movement and to prevent relative axial movement. The button may be axially displaceable relative to the housing and may comprise teeth engaging with corresponding teeth of the housing, preferably of the first shell, depending on the relative axial position of the button to the housing. In addition or as an alternative, the dose setting grip may be clipped to the housing, e.g. to the second shell, to allow relative rotational movement and to prevent relative axial movement. One end of the drive spring and/or one end of the clutch spring may be axially and/or rotationally constrained to the housing, e.g. to the first shell. The gauge element may be rotationally constrained to the housing and may be axially displaceably guided in the housing, e.g. in the second shell.

The method for producing a housing with a twin-shot injection moulded casing comprises the steps of injection-moulding during a first shot the first shell in a translucent material and thereafter injection-moulding during a second shot the second shell in an opaque material. Preferably, the at least one window in the housing is formed in any areas where the second shot does not cover the first shot.

As an alternative, a window could be formed by moulding the opaque shot inside the transparent shot. Even if the opaque shot is outside the transparent shot, the opaque shot could be moulded first.

Injection moulding of the housing in two or more shots may include providing one or more stakes, e.g. on the element that is formed during the first shot, to hold the moulded element in place during a subsequent moulding shot. Such stakes could further be used as a window of the housing, if the stakes are formed by a translucent or transparent material.

The term “medicament”, as used herein, means a pharmaceutical formulation containing at least one pharmaceutically active compound,

wherein in one embodiment the pharmaceutically active compound has a molecular weight up to 1500 Da and/or is a peptide, a protein, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(w-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 Exendin-4(1-39),

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group-Lys6-NH2 may be bound to the C-terminus of the Exendin-4 derivative;

or an Exendin-4 derivative of the sequence

des Pro36 Exendin-4(1-39)-Lys6-NH2 (AVE0010),

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2, des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; a and y contain approximately 450 amino acids and δ approximately 500 amino acids, while μ and ε have approximately 550 amino acids. Each heavy chain has two regions, the constant region (CH) and the variable region (VH). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL).

The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H-H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

### BRIEF DESCRIPTION OF THE DRAWINGS

Non-limiting, exemplary embodiments of the disclosure will now be described with reference to the accompanying drawings, in which:

FIG. 1 shows a top view of a drug delivery device according to a first embodiment of the present disclosure;

FIG. 2 shows an exploded view of the components of the device of FIG. 1;

FIG. 3 shows a top view of a first shell of the housing of the device of FIG. 1;

FIG. 4 shows a top view of a second shell of the housing of the device of FIG. 1;

FIG. 5 shows a sectional view of the housing of the device of FIG. 1;

FIG. 6 shows a detail of FIG. 5;

FIG. 7 shows a detail of the housing of FIG. 1 with a button; and

FIG. 8 shows an alternative to the detail of FIG. 7.

### DETAILED DESCRIPTION

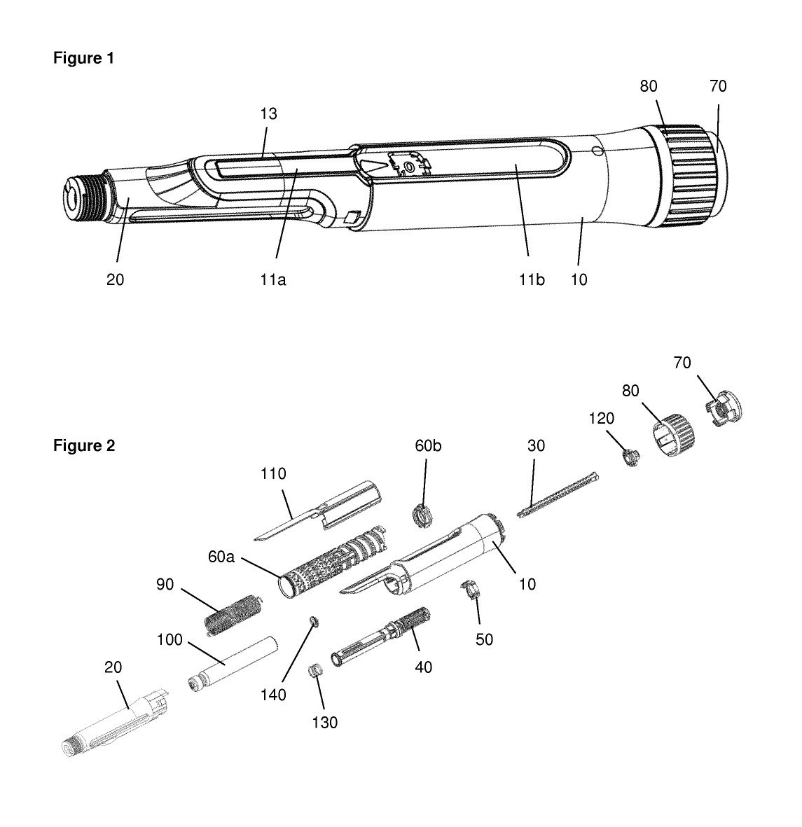

FIG. 1 shows a drug delivery device in the form of an injection pen. The device has a distal end (left end in FIG. 1) and a proximal end (right end in FIG. 1). The component parts of the drug delivery device are shown in FIG. 2. The drug delivery device comprises a body or housing 10, a cartridge holder 20, a lead screw (piston rod) 30, a drive sleeve 40, a nut 50, a dose indicator (number sleeve) 60, a button 70, a dial grip or dose selector 80, a torsion spring 90, a cartridge 100, a gauge element 110, a clutch plate 120, a clutch spring 130 and a bearing 140. A needle arrangement (not shown) with a needle hub and a needle cover may be provided as additional components, which can be exchanged as explained above. All components are located concentrically about a common principal axis of the mechanism.

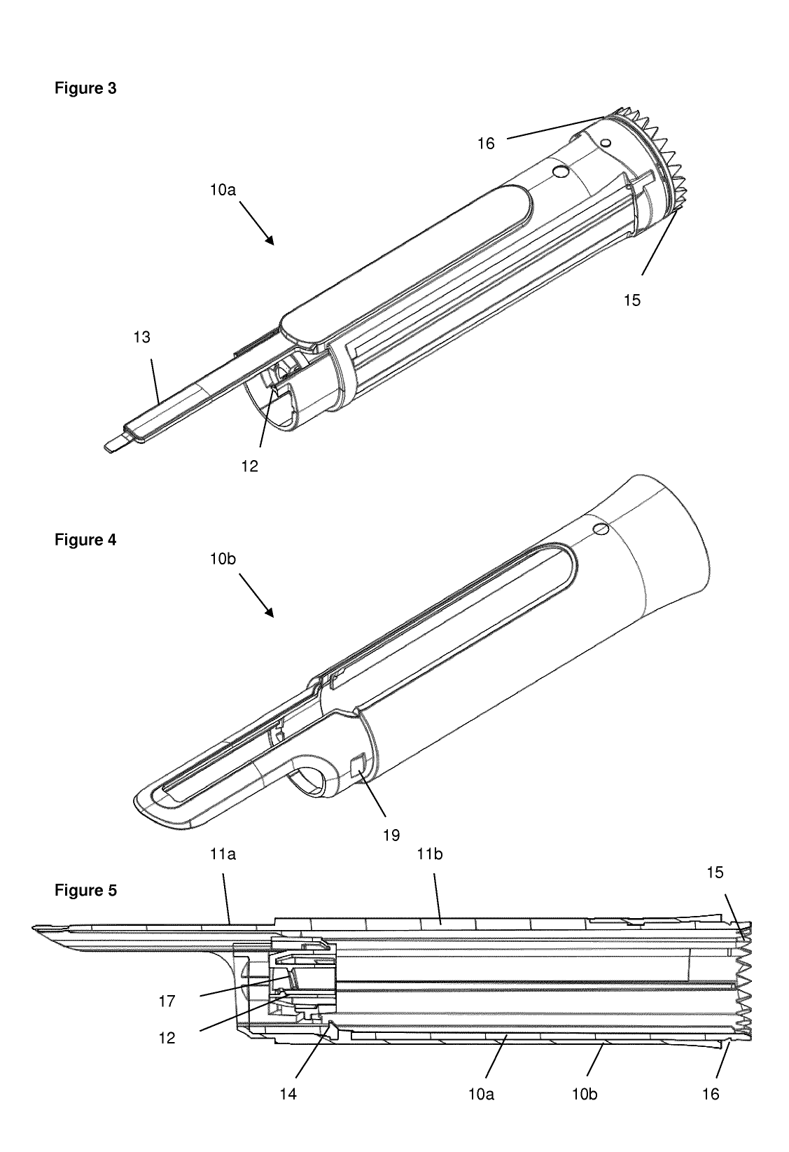

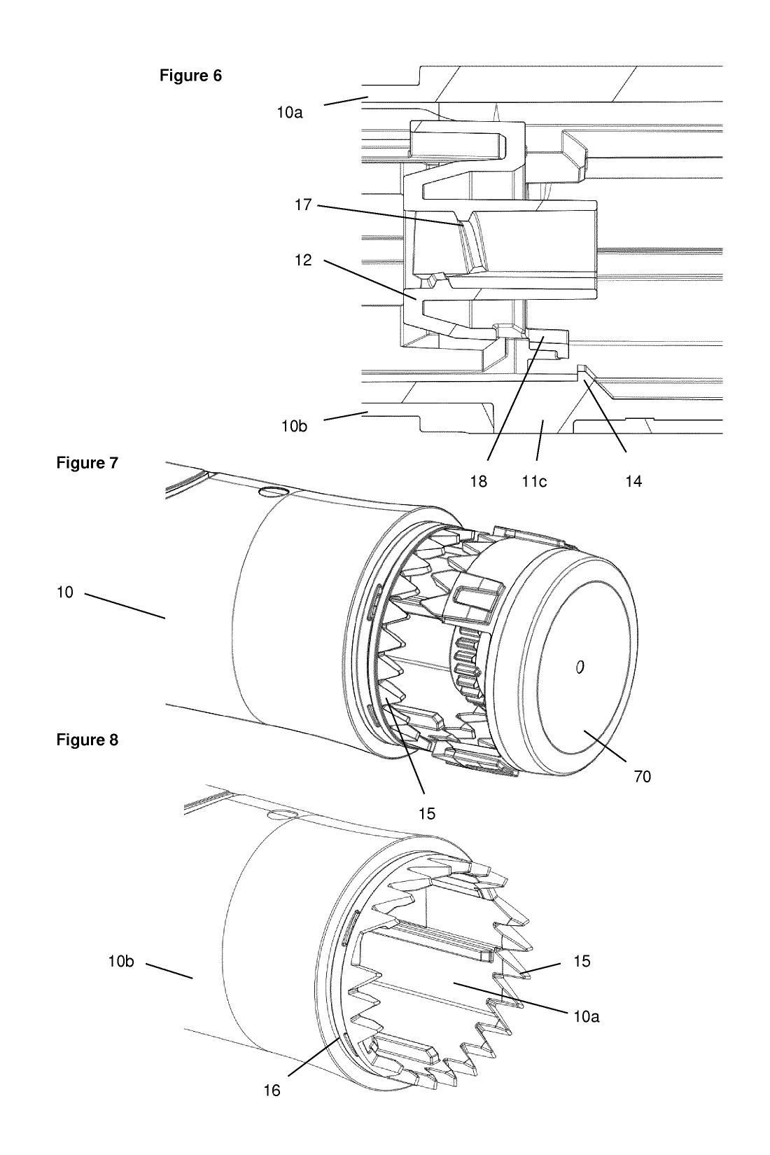

The housing 10 or body is a generally tubular casing element having a proximal end with an enlarged diameter. The housing 10 provides location for the liquid medication cartridge 100 and cartridge holder 20. As shown in FIGS. 3 and 4, the housing comprises a casing consisting of a first shell 10a and a second shell 10b which are injection moulded using a twin-shot injection technique. In this embodiment, a first window 11a, a second window (or lens) 11b and a third window 11c (FIG. 6) are incorporated into the housing body by twin-shot molding. The windows 11a, 11b, 11c are molded during a first shot in a translucent (and preferably transparent) material, and the outer cover or second shell 10b of the housing is molded during a second shot in an opaque material.

Openings in the second shell 10b allow the user to view internal parts of the injector through the first shell 10a. For examples a part that may be viewed through at least one of these windows 11a, 11b, 11c is the number sleeve 60 which may be printed with dose numbers, allowing the user to determine the size of the selected dose. Further, at least one of these windows 11a, 11b, 11c may be used to view the gauge element 110. The gauge element 110 is used to create a sliding scale or progress bar. As a dose is set by the user, the gauge element 110 translates axially, the distance moved proportional to the magnitude of the dose set. This feature gives clear feedback to the user regarding the approximate size of the dose set. The gauge element 110 also provides feedback to the user during dispense regarding dispense progress without the need to read the dose number itself. These windows 11a, 11b, 11c reduce dust ingress and prevent the user from touching moving parts may be separate components. A first shot of translucent material forms the internal features and the windows (FIG. 3), and then a second shot of opaque material forms the outer cover of the housing 10 (FIG. 4). In this way, a window is formed in any areas where the second shot (second shell 10b) does not cover the first shot (first shell 10a).

An e.g. flange-like insert (interface) 12 is shown in FIG. 6 as a part of the first shell 10a. This insert is moulded together with the first shell 10a in the translucent material. As an alternative, the insert or parts thereof may be formed with the second shell 10b in the opaque material.

Internal features (interfaces) may be molded as part of the first shell 10a, or as part of the second shell 10b in any areas where the first shell 10a extends through to the inside surfaces of the housing 10. In the exemplary embodiment shown in the Figures, the distal end of the first shell 10a is provided with an axially extending strip 13 partly overlapping cartridge holder 20. This strip forms the first window 11a. The housing 10 further comprises a fixture for engaging number sleeve 60. This fixture has the form of an inner bead 14 (FIG. 6) which is located at a distal region of housing 10 on its inner surface. Further, angled crown teeth 15 are molded at the proximal end of the first shell 10a. The first shell 10a has a feature on its external surface, e.g. a circumferential groove 16, to axially retain the dose selector 80.

The insert 12 of the first shell 10a may have various interfaces. For example, the tubular inner body of insert 12 comprises an inner thread 17 engaging the piston rod 30. In addition the radial space between the tubular inner body and the outer portion of the first shell 10a may provide a bearing area receiving the drive spring 90 and/or clutch spring 130. Further, teeth 18 are provided on the insert 12 of the first shell 10a. Teeth 18 interact with drive member 40 to rotationally couple and de-couple the drive member and the housing 10.

The first shell 10a and/or the second shell 10b has at least one internal, axially orientated slot (hidden in FIG. 4) or the like for axially guiding the gauge element 110. Further, ramps 19 are provided on the second shell 10b for snap attachment of a cap (not shown).

The Figures depict the housing 10 as a single housing component. However, the housing 10 could comprise two or more housing components which may be permanently attached to each other during assembly of the device.

The cartridge holder 20 is located at the distal side of housing 10 and permanently attached thereto. The cartridge holder may be a transparent or translucent component which is tubular to receive cartridge 100. The distal end of cartridge holder 20 may be provided with means for attaching a needle arrangement. A removable cap (not shown) may be provided to fit over the cartridge holder 20 and may be retained via clip features on the housing 10.

The piston rod 30 is rotationally constrained to the drive sleeve 40 via a splined interface. When rotated, the piston rod 30 is forced to move axially relative to the drive sleeve 40, through its threaded interface with the insert 12 of housing 10. The lead screw 30 is an elongate member with an outer thread engaging the corresponding thread of the insert 12 of housing 10. The interface comprises at least one longitudinal groove or track and a corresponding protrusion or spline of the driver 40. At its distal end, the lead screw 30 is provided with an interface for clip attachment of the bearing 140.

The drive sleeve 40 is a hollow member surrounding the lead screw 30 and arranged within number sleeve 60. It extends from an interface with the clutch plate 120 to the contact with the clutch spring 130. The drive sleeve 40 is axially movable relative to the housing 10, the piston rod 30 and the number sleeve 60 in the distal direction against the bias of clutch spring 130 and in the opposite proximal direction under the bias of clutch spring 130.

A splined tooth interface 18 with the housing 10 prevents rotation of the drive sleeve 40 during dose setting. This interface comprises a ring of radially extending outer teeth at the distal end of drive sleeve 40 and corresponding radially extending inner teeth of the housing component 10. When the button 70 is pressed, these drive sleeve 40 to housing 10 spline teeth are disengaged allowing the drive sleeve 40 to rotate relative to housing 10. A further splined tooth interface with the number sleeve 60 is not engaged during dialling, but engages when the button 70 is pressed, preventing relative rotation between the drive sleeve 40 and number sleeve 60 during dispense. In a preferred embodiment this interface comprises inwardly directed splines on a flange on the inner surface of the number sleeve 60 and a ring of radially extending outer splines of drive sleeve 40. These corresponding splines are located on the number sleeve 60 and the drive sleeve 40, respectively, such that axial movement of the drive sleeve 40 relative to the (axially fixed) number sleeve 60 engages or disengages the splines to rotationally couple or decouple the drive sleeve 40 and the number sleeve 60.

A further interface of the drive sleeve 40 comprises a ring of ratchet teeth located at the proximal end face of drive sleeve 40 and a ring of corresponding ratchet teeth on the clutch plate 120.

The driver 40 has a threaded section providing a helical track for the nut 50. In addition, a last dose abutment or stop is provided which may be the end of the thread track or preferably a rotational hard stop for interaction with a corresponding last dose stop of nut 50, thus limiting movement of the nut 50 on the driver thread. At least one longitudinal spline of the driver 40 engages a corresponding track of the lead screw 30.

The last dose nut 50 is located between the number sleeve 60 and the drive sleeve 40. It is rotationally constrained to the number sleeve 60, via a splined interface. It moves along a helical path relative to the drive sleeve 40, via a threaded interface, when relative rotation occurs between the number sleeve 60 and drive sleeve 40 which is during dialling only. As an alternative, the nut 50 may be splined to the driver 40 and threaded to the number sleeve 60. A last dose stop is provided on nut 50 engaging a stop of drive sleeve 40 when a dose is set corresponding to the remaining dispensable amount of medicament in the cartridge 100.

The dose indicator or number sleeve 60 is a tubular element. The number sleeve 60 is rotated during dose setting (via dose selector 80) and dose correction and during dose dispensing by torsion spring 90. Together with gauge element 110 the number sleeve 60 defines a zero position (‘at rest’) and a maximum dose position. Thus, the number sleeve 60 may be seen as a dose setting member.

For manufacturing reasons, the number sleeve 60 of the embodiment shown in the Figures comprises a number sleeve lower 60a which is rigidly fixed to a number sleeve upper 60b during assembly to form the number sleeve 60. Number sleeve lower 60a and number sleeve upper 60b are separate components only to simplify number sleeve 60 mould tooling and assembly. As an alternative, the number sleeve 60 may be a unitary component. The number sleeve 60 is constrained to the housing 10 by snap engagement to allow rotation but not translation. The number sleeve 60 comprises an annular recess or groove near its distal end which engages a corresponding bead 14 on an inner surface of the housing 10. The number sleeve lower 60a is marked with a sequence of numbers, which are visible through the gauge element 110 and the openings 11a, 11b in the housing 10, to denote the dialled dose of medicament.

Further, the number sleeve lower 60a has a portion with an outer thread engaging the gauge element 110. End stops are provided at the opposite ends of thread to limit relative movement with respect to the gauge element 110.

Clutch features which have the form of a ring of splines are provided inwardly directed on number sleeve upper 60b for engagement with splines of the button 70 during dose setting and dose correction. A clicker arm is provided on the outer surface of number sleeve 60 which interacts with the drive sleeve 40 and the gauge member 110 for generating a feedback signal. In addition, the number sleeve lower 60a is rotationally constrained to the nut 50 and to the clutch plate 120 via a splined interface comprising at least one longitudinal spline. Further, number sleeve lower 60a comprises an interface for attachment of the torsion spring 90.

The button 70 which forms the proximal end of the device is permanently splined to the dose selector 80. A central stem extends distally from the proximal actuation face of the button 70. The stem is provided with a flange carrying the splines for engagement with splines of the number sleeve upper 60b. Thus, it is also splined via splines to the number sleeve upper 60b when the button 70 is not pressed, but this spline interface is disconnected when the button 70 is pressed. The button 70 has a discontinuous annular skirt with splines. When the button 70 is pressed, splines on the button 70 engage with splines on the housing 10, preventing rotation of the button 70 (and hence the dose selector 80) during dispense. These splines disengage when the button 70 is released, allowing a dose to be dialled. Further, a ring of ratchet teeth is provided on the inner side of button flange for interaction with clutch plate 120.

The dose selector 80 is axially constrained to the housing 10. It is rotationally constrained, via the splined interface, to the button 70. This splined interface which includes grooves interacting with spline features formed by the annular skirt of button 70 remains engaged irrespective of the dose button 70 axial positions. The dose selector 80 or dose dial grip is a sleeve-like component with a serrated outer skirt.

The torsion spring 90 is attached at its distal end to the housing 10 and at the other end to the number sleeve 60. The torsion spring 90 is located inside the number sleeve 60 and surrounds a distal portion of the drive sleeve 40. The torsion spring 90 is pre-wound upon assembly, such that it applies a torque to the number sleeve 60 when the mechanism is at zero units dialled.

The action of rotating the dose selector 80, to set a dose, rotates the number sleeve 60 relative to the housing 10, and charges the torsion spring 90 further.

The cartridge 100 is received in cartridge holder 20. The cartridge 100 may be a glass ampoule having a moveable rubber bung at its proximal end. The distal end of cartridge 100 is provided with a pierceable rubber seal which is held in place by a crimped annular metal band. In the embodiment depicted in the Figures, the cartridge 100 is a standard 1.5 ml cartridge. The device is designed to be disposable in that the cartridge 100 cannot be replaced by the user or health care professional. However, a reusable variant of the device could be provided by making the cartridge holder 20 removable and allowing backwinding of the lead screw 30 and the resetting of nut 50.

The gauge element 110 is constrained to prevent rotation but allow translation relative to the housing 10 via a splined interface. The gauge element 110 has a helical feature on its inner surface which engages with the helical thread cut in the number sleeve 60 such that rotation of the number sleeve 60 causes axial translation of the gauge element 110. This helical feature on the gauge element 110 also creates stop abutments against the end of the helical cut in the number sleeve 60 to limit the minimum and maximum dose that can be set.

The gauge element 110 has a generally plate or band like component having a central aperture or window and two flanges extending on either side of the aperture. The flanges are preferably not transparent and thus shield or cover the number sleeve 60, whereas the aperture or window allows viewing a portion of the number sleeve lower 60a. Further, gauge element 110 has a cam and a recess interacting with the clicker arm of the number sleeve 60 at the end of dose dispensing.

The clutch plate 120 is a ring-like component. The clutch plate 120 is splined to the number sleeve 60 via splines. It is also coupled to the drive sleeve 40 via a ratchet interface. The ratchet provides a detented position between the number sleeve 60 and drive sleeve 40 corresponding to each dose unit, and engages different ramped tooth angles during clockwise and anti-clockwise relative rotation. A clicker arm is provided on the clutch plate 120 for interaction with ratchet features of the button 70.

The clutch spring 130 is a compression spring. The axial position of the drive sleeve 40, clutch plate 120 and button 70 is defined by the action of the clutch spring 130, which applies a force on the drive sleeve 40 in the proximal direction. This spring force is reacted via the drive sleeve 40, clutch plate 120, and button 70, and when ‘at rest’ it is further reacted through the dose selector 80 to the housing 10. The spring force ensures that the ratchet interface between drive sleeve 40 and clutch plate 120 is always engaged. In the ‘at rest’ position, it also ensures that the button splines are engaged with the number sleeve splines, and the drive sleeve teeth are engaged with teeth of the housing 10.

The bearing 140 is axially constrained to the piston rod 30 and acts on the bung within the liquid medicament cartridge. It is axially clipped to the lead screw 30, but free to rotate.

With the device in the ‘at rest’ condition as shown in FIG. 1, the number sleeve 60 is positioned against its zero dose abutment with the gauge element 110 and the button 70 is not depressed. Dose marking ‘0’ on the number sleeve 60 is visible through the window 11b of the housing 10 and gauge element 110, respectively.

The torsion spring 90, which has a number of pre-wound turns applied to it during assembly of the device, applies a torque to the number sleeve 60 and is prevented from rotating by the zero dose abutment.

The user selects a variable dose of liquid medicament by rotating the dose selector 80 clockwise, which generates an identical rotation in the number sleeve 60. Rotation of the number sleeve 60 causes charging of the torsion spring 90, increasing the energy stored within it. As the number sleeve 60 rotates, the gauge element 110 translates axially due to its threaded engagement thereby showing the value of the dialled dose. The gauge element 110 has flanges either side of the window area which cover the numbers printed on the number sleeve 60 adjacent to the dialled dose to ensure only the set dose number is made visible to the user.

A specific feature of this disclosure is the inclusion of a visual feedback feature in addition to the discrete dose number display typical on devices of this type. The distal end of the gauge element 110 creates a sliding scale through the small window 11a in the housing 10. As an alternative, the sliding scale could be formed using a separate component engaged with the number sleeve 60 on a different helical track.

As a dose is set by the user, the gauge element 110 translates axially, the distance moved proportional to the magnitude of the dose set. This feature gives clear feedback to the user regarding the approximate size of the dose set. The dispense speed of an auto-injector mechanism may be higher than for a manual injector device, so it may not be possible to read the numerical dose display during dispense. The gauge feature provides feedback to the user during dispense regarding dispense progress without the need to read the dose number itself. For example, the gauge display may be formed by an opaque element on the gauge element 110 revealing a contrasting coloured component underneath. Alternatively, the revealable element may be printed with coarse dose numbers or other indices to provide more precise resolution. In addition, the gauge display simulates a syringe action during dose set and dispense.

The drive sleeve 40 is prevented from rotating as the dose is set and the number sleeve 60 rotated, due to the engagement of its splined teeth with teeth of the housing 10. Relative rotation must therefore occur between the clutch plate 120 and drive sleeve 40 via the ratchet interface.

The user torque required to rotate the dose selector 80 is a sum of the torque required to wind up the torsion spring 90, and the torque required to overhaul the ratchet interface. The clutch spring 130 is designed to provide an axial force to the ratchet interface and to bias the clutch plate 120 onto the drive sleeve 40. This axial load acts to maintain the ratchet teeth engagement of the clutch plate 120 and drive sleeve 40. The torque required to overhaul the ratchet in the dose set direction is a function of the axial load applied by the clutch spring 130, the clockwise ramp angle of the ratchet teeth, the friction coefficient between the mating surfaces and the mean radius of the ratchet interface.

As the user rotates the dose selector 80 sufficiently to increment the mechanism by one increment, the number sleeve 60 rotates relative to the drive sleeve 40 by one ratchet tooth. At this point the ratchet teeth re-engage into the next detented position. An audible click is generated by the ratchet re-engagement, and tactile feedback is given by the change in torque input required.

Relative rotation of the number sleeve 60 and the drive sleeve 40 is allowed. This relative rotation also causes the last dose nut 50 to travel along its threaded path, towards its last dose abutment on the drive sleeve 40.

With no user torque applied to the dose selector 80, the number sleeve 60 is now prevented from rotating back under the torque applied by the torsion spring 90, solely by the ratchet interface between the clutch plate 120 and the drive sleeve 40. The torque necessary to overhaul the ratchet in the anti-clockwise direction is a function of the axial load applied by the clutch spring 130, the anti-clockwise ramp angle of the ratchet, the friction coefficient between the mating surfaces and the mean radius of the ratchet features. The torque necessary to overhaul the ratchet must be greater than the torque applied to the number sleeve 60 (and hence clutch plate 120) by the torsion spring 90. The ratchet ramp angle is therefore increased in the anti-clockwise direction to ensure this is the case whilst ensuring the dial-up torque is as low as possible.

The user may now choose to increase the selected dose by continuing to rotate the dose selector 80 in the clockwise direction. The process of overhauling the ratchet interface between the number sleeve 60 and drive sleeve 40 is repeated for each dose increment. Additional energy is stored within the torsion spring 90 for each dose increment and audible and tactile feedback is provided for each increment dialled by the re-engagement of the ratchet teeth. The torque required to rotate the dose selector 80 increases as the torque required to wind up the torsion spring 90 increases. The torque required to overhaul the ratchet in the anti-clockwise direction must therefore be greater than the torque applied to the number sleeve 60 by the torsion spring 90 when the maximum dose has been reached.

If the user continues to increase the selected dose until the maximum dose limit is reached, the number sleeve 60 engages with its maximum dose abutment on the maximum dose abutment of gauge element 110. This prevents further rotation of the number sleeve 60, clutch plate 120 and dose selector 80.

Depending on how many increments have already been delivered by the mechanism, during selection of a dose, the last dose nut 50 may contact its last dose abutment with stop face of the drive sleeve 40. The abutment prevents further relative rotation between the number sleeve 60 and the drive sleeve 40, and therefore limits the dose that can be selected. The position of the last dose nut 50 is determined by the total number of relative rotations between the number sleeve 60 and drive sleeve 40, which have occurred each time the user sets a dose.

With the mechanism in a state in which a dose has been selected, the user is able to deselect any number of increments from this dose. Deselecting a dose is achieved by the user rotating the dose selector 80 anti-clockwise. The torque applied to the dose selector 80 by the user is sufficient, when combined with the torque applied by the torsion spring 90, to overhaul the ratchet interface between the clutch plate 120 and drive sleeve 40 in the anti-clockwise direction. When the ratchet is overhauled, anti-clockwise rotation occurs in the number sleeve 60 (via the clutch plate 120), which returns the number sleeve 60 towards the zero dose position, and unwinds the torsion spring 90. The relative rotation between the number sleeve 60 and drive sleeve 40 causes the last dose nut 50 to return along its helical path, away from the last dose abutment.

With the mechanism in a state in which a dose has been selected, the user is able to activate the mechanism to commence delivery of a dose. Delivery of a dose is initiated by the user depressing the button 70 axially in the distal direction.

When the button 70 is depressed, splines between the button 70 and number sleeve 60 are disengaged, rotationally disconnecting the button 70 and dose selector 80 from the delivery mechanism, i.e. from number sleeve 60, gauge element 110 and torsion spring 90. Splines on the button 70 engage with splines on the housing 10, preventing rotation of the button 70 (and hence the dose selector 80) during dispense. As the button 70 is stationary during dispense, it can be used in the dispense clicker mechanism. A stop feature in the housing 10 limits axial travel of the button 70 and reacts any axial abuse loads applied by the user, reducing the risk of damaging internal components.

The clutch plate 120 and drive sleeve 40 travel axially with the button 70. This engages the splined tooth interface between the drive sleeve 40 and number sleeve 60, preventing relative rotation between the drive sleeve 40 and number sleeve 60 during dispense. The splined tooth interface between the drive sleeve 40 and the housing 10 disengages, so the drive sleeve 40 can now rotate and is driven by the torsion spring 90 via the number sleeve 60, and clutch plate 120.

Rotation of the drive sleeve 40 causes the piston rod 30 to rotate due to their splined engagement, and the piston rod 30 then advances due to its threaded engagement to the housing 10. The number sleeve 60 rotation also causes the gauge element 110 to traverse axially back to its zero position whereby the zero dose abutment stops the mechanism.

Tactile feedback during dose dispense is provided via the compliant cantilever clicker arm integrated into the clutch plate 120. This arm interfaces radially with ratchet features on the inner surface of the button 70, whereby the ratchet tooth spacing corresponds to the number sleeve 60 rotation required for a single increment dispense. During dispense, as the number sleeve 60 rotates and the button 70 is rotationally coupled to the housing 10, the ratchet features engage with the clicker arm to produce an audible click with each dose increment delivered.

Delivery of a dose continues via the mechanical interactions described above while the user continues to depress the button 70. If the user releases the button 70, the clutch spring 130 returns the drive sleeve 40 to its ‘at rest’ position (together with the clutch plate 120 and button 70), engaging the splines between the drive sleeve 40 and housing 10, preventing further rotation and stopping dose delivery.

During delivery of a dose, the drive sleeve 40 and number sleeve 60 rotate together, so that no relative motion in the last dose nut 50 occurs. The last dose nut 50 therefore travels axially relative to the drive sleeve 40 during dialling only.

Once the delivery of a dose is stopped, by the number sleeve 60 returning to the zero dose abutment, the user may release the button 70, which will re-engage the spline teeth between the drive sleeve 40 and housing 10. The mechanism is now returned to the ‘at rest’ condition.

At the end of dose dispensing, additional audible feedback is provided in the form of a ‘click’, distinct from the ‘clicks’ provided during dispense, to inform the user that the device has returned to its zero position via the interaction of the clicker arm on the number sleeve 60 with the ramp on the drive sleeve 40 and the cam and the recess on the gauge element 110. This embodiment allows feedback to only be created at the end of dose delivery and not created if the device is dialled back to, or away from, the zero position.

FIGS. 7 and 8 show two alternatives of an interface between the housing 10, namely the first shell 10a of the housing, and the button 70 or trigger used to initiate dose dispensing. For example, angled crown teeth 15 are molded at the proximal end of the first shell 10a. When the button 70 is depressed, spline teeth on the button 70 engage with spline teeth 15 on the housing, preventing rotation of the button during dispense, and also preventing rotation of the dial grip 80, as this is splined to the button. The form of these teeth makes them easy to mold, as they extend through the thickness of the housing 10. It also pushes the button 70 out of engagement if dial grip 80 is turned when the button 70 is pressed.

During selection and deselection of a dose, a clutch interface between the drive sleeve 40 and clutch plate 120 is able to slip, allowing relative rotation between the two parts. The clutch plate 120 is rotationally constrained to the number sleeve 60, so the clutch allows the number sleeve and drive sleeve to rotate relative to one another. When the button 70 is fully pressed, the clutch spring 130 becomes more compressed, reducing the axial movement that is possible between the drive sleeve 40 and clutch plate 120, and so preventing slipping of the clutch interface. When the button 70 is only partially pressed, there is a risk that the clutch may slip. The number sleeve 60 would then rotate relative to the drive sleeve 40, driven by the torsion spring 90, and the user would not receive the whole of the selected dose. As mentioned above, this problem may be solved by splines between the number sleeve 60 and drive sleeve 40 that engage before dispense can start.

FIG. 8 shows an alternative solution in which each crown tooth 15 between the housing 10 and button 70 has one angled face and one flat face. When the button is partially pressed, the teeth 15 engage between the housing 10 and button 70. The flat sides of the teeth 15, in addition to splines between the button 60 and number sleeve 60, prevent rotation of the number sleeve 60 in the dispense direction. The splined tooth interface 18 between the drive sleeve 40 and the housing 10 remains engaged, so the drive sleeve 40 cannot rotate and there is no relative rotation between the drive sleeve 40 and the clutch plate 120. When the button 70 is pressed further, the splined tooth interface 18 between the drive sleeve 40 and the housing 10 disengages. As there is no torque on the drive sleeve 40, it does not rotate. The splines between the button 70 and number sleeve 60 then disengage, rotationally disconnecting the number sleeve 60 from the housing 10, so the drive sleeve 40 can rotate to deliver the dose. The number sleeve 60, clutch plate 120 and drive sleeve 40 rotate together, driven by the torsion spring 90.

### REFERENCE NUMERALS

10 housing (casing)

10a first shell

10b second shell

11a-c window

12 insert (interface)

13 strip

14 bead

15 crown teeth

16 groove

17 thread

18 teeth

19 ramp

20 cartridge holder

30 piston rod (lead screw)

40 drive sleeve

50 nut

60 dose setting element

60a number sleeve lower

60b number sleeve upper

70 button

80 dose selector

90 torsion spring

100 cartridge

110 gauge element

120 clutch spring

130 clutch spring

140 bearing

1. A housing for a drug delivery device, the housing comprising:
a twin-shot injection-molded casing with at least one window and at least one interface for engaging a further component part of the drug delivery device, wherein the casing comprises a first shell injection-molded translucent material and a second shell injection-molded in an opaque material.

2. The housing according to claim 1, wherein the first shell and the second shell are permanently joined with each other.

3. The housing according to claim 1, wherein the second shell at least partly surrounds the first shell.

4. The housing according to claim 1, wherein the first shell comprises the at least one window and the at least one interface for engaging a further component part of the drug delivery device.

5. The housing according to claim 1, wherein the second shell comprises the at least one interface for engaging a further component part of the drug delivery device.

6. The housing according to claim 5, wherein the at least one interface for engaging a further component part of the drug delivery device comprises a thread, a rib, a groove, a bead, a tooth, a ramp and/or an arm.

7. The housing according to a claim 1, wherein the first shell comprises a tubular portion with an axially extending protrusion at a distal end of the tubular portion and a series of crown teeth at an opposite, proximal end of the tubular portion.

8. The housing according to claim 7, wherein the tubular portion comprises an oblong elevation extending in an axial direction, and the oblong elevation forms the at least one window.

9. The housing according to claim 7, wherein the first shell further comprises an insert located at or near the distal end of the tubular portion.

10. The housing according to claim 1, wherein the second shell comprises ramps and at least one guiding rib or groove.

11. A drug delivery device for selecting and dispensing a number of user variable doses of a medicament, the device comprising:
a housing comprising a twin-shot injection-molded casing with at least one window and at least one interface for engaging a further component part of the drug delivery device, wherein the casing comprises a first shell injection-molded in a translucent material and a second shell injection-molded in an opaque material; and
a cartridge holder and a cartridge containing a medicament disposed in the housing.

12. The drug delivery device according to claim 11, wherein the housing is coupled via the at least one interface to at least one of:
the cartridge holder,
a piston rod,
a drive member,
a nut,
a dose setting element,
a button,
a dose setting grip,
a drive spring,
a gauge element,
a clutch, and
a clutch spring.

13. The drug delivery device according to claim 12, wherein
the piston rod is in threaded engagement with the housing,
the drive member is axially displaceable relative to the housing and comprises teeth engaging with corresponding teeth of the housing depending on the relative axial position of the drive member to the housing,
the dose setting element is clipped to the housing to allow relative rotational movement and to prevent relative axial movement,
the button is axially displaceable relative to the housing and comprises teeth engaging with corresponding teeth of the housing depending on the relative axial position of the button to the housing,
the dose setting grip is clipped to the housing to allow relative rotational movement and to prevent relative axial movement,
one end of the drive spring is axially and/or rotationally constrained to the housing,
the gauge element is rotationally constrained to the housing and axially displaceably guided in the housing, and/or
one end of the clutch spring is axially and/or rotationally constrained to the housing.

14. A method for producing a housing for a drug delivery device, the method comprising:
injection molding a first shell during a first shot with a translucent material; and
injection molding a second shell during a second shot with an opaque material,
wherein the first and second shells form at least one window and at least one interface for engaging a further component part of the drug delivery device.

15. The method according to claim 14, wherein the at least one window is formed in an area in which the opaque material does not cover the translucent material.

16. The drug delivery device of claim 11, wherein the medicament comprises a pharmaceutically active compound.
